(19) United States
(12) Patent Application Publication
(10) Pub. No.: US 2010/0330000 A1
(43) Pub. Date: Dec. 30, 2010

(54) ENZYMES FOR TREATMENT OF GINGIVITIS

(76) Inventors: Kristian Hellgren, Viken (SE); Max Rutman, Santiago (CL); Jan Vincent, Stockholm (SE)

Correspondence Address: KNOBLE, YOSHIDA & DUNLEAVY, EIGHT PENN CENTER, SUITE 1350, 1628 JOHN F KENNEDY BLVD, PHILADELPHIA, PA, 19103, US

(21) Appl. No.: 12/741,781

(22) PCT Filed: Nov. 5, 2008
(86) PCT No.: PCT/EP2008/065029

§ 371 Date: Sep. 3, 2010

Related U.S. Application Data
(60) Provisional application No. 60/985,860, filed on Nov. 6, 2007.

(51) Int. Cl. A61K 9/68; A61P 1/02

(52) U.S. Cl. 424/48

(57) ABSTRACT

The invention relates to a method for treating, controlling and/or preventing diseases of the oral cavity, particularly diseases and disorders induced accumulation of plaque, by administering a chewing gum composition comprising at least one krill enzyme. Specifically, the invention is directed to a method for preventing, treating and controlling the development of gingivitis, gum disease and symptoms thereof using a composition having a krill enzyme concentration of about 0.005 U/g to about 0.05 U/g of the chewing gum composition.

### BACKGROUND OF THE INVENTION

1. Field of the Invention

The invention relates to a method for treating, controlling and/or preventing ailments of the oral cavity such as aphtes, lesions, gum disease and dental diseases. Specifically, the invention pertains to a method for treating ailments induced by accumulation of plaque such as gingivitis, gum diseases, and/or symptoms thereof.

2. Description of the Related Technology

Controlling the accumulation dental plaque is an important prophylactic measure for maintaining good dental health. Dental plaque is an organized film of oral bacteria and organic matrix that causes various oral diseases such as caries and periodontitis. Accumulation of dental plaque at the gingival margin causes periodontal inflammation, i.e. gingivitis, mainly through the release of toxic bacterial products. If left undisturbed, the dental plaque proliferates into the gingival ulcus and develops an anaerobic bacterial composition where Gram-negative rods dominate. While subgingival plaque causes periodontal disease, the supragingival plaque, which mainly contains Gram-positive bacteria, induces caries via its extensive acid production upon sugar intake.

In order to treat, control or prevent such problems, significant research has been directed to compositions which disrupt plaque build-up. Although a number of agents have shown promising antiplaque activity in vitro, few substances are currently used in vivo. With the exception of chlorhexidine, triclosan or amino alcohols, most agents have proven insufficient as anti-plaque agents. Additionally, many of these agents also induce the complete elimination of microflora from the oral cavity. The mouth like other areas of the digestive tract possesses a natural microflora whose presence confers several beneficial properties to the host. Therefore, there exists a need to develop an antiplaque agent that controls rather than eliminates dental plaque by limiting the development of the oral dental bio film in order to avoid concurrent elimination of desirable natural microflora.

Recently, enzymes have been considered as potentially interesting anti-plaque agents because of their ability to dissolve organic debris and their biocompatibility. Preliminary studies, however, indicate that due to a lack of sustained enzyme retention in the oral cavity and/or insufficient enzymatic activity, enzymes produced disappointing results.

Of the various enzymes studied, krill derived enzymes, such as those incorporated in Krillase®, have shown some promise for dental applications. Krillase® originates from the digestive tract of a shrimp-like animal, Antarctic krill (Euphausia superba). The ecological niche occupied by Antarctic krill implies that this group of crustaceans has an exceptionally effective digestive apparatus containing a co-operative multi-enzyme system involving both endo- and exopeptidases. In addition, these enzymes have much lower activation energies than those of mammalian enzymes ensuring highly efficient breakdown of diverse biological substrates (Hellgren et al, 1999). Previous studies have proven that Krillase® is both quantitatively and qualitatively effective on plaque/bio film as well as on bacterial adherence to teeth surfaces, causing significant reduction in plaque accumulation (Hahn-Berg C I, “Properties of interfacial proteinaceous films with emphasis on oral systems” PhD thesis, Inst. Surface Chem., Stockholm and Dept. Food Technol., Lund University, Lund, 2003; Hahn Berg C, Kalfas S, Malmsten M, Arnebrant T “Proteolytic degradation of oral bio films in vitro and in vivo: potential of proteases originating from Euphausia superba for plaque control” Eur J Oral Sci 109, 316-324, 2001).

Due to their ability to rapidly degrade biological contaminants in alkaline, neutral or acid media, krill enzymes are known to be useful for treating gum infections and dental plaque build-up. For example, WO Publication No. 93/24142 discloses a method for treating acute or chronic gum infections and inflammation of the gums using a krill enzyme composition wherein the krill enzyme was present at a concentration of about 5 Casein-Units/ml of solution. Patients were administered the composition as a mouth rinse and were required to rinse with the composition three times a day for about 1 week. The desirable dosage range was determined to be about 0.1 to 100 mg or 1 to 35 mg per treatment. WO Publication No. 93/24142 also discloses a treatment for dental plaque using a composition comprising krill enzyme at a concentration of about 5 Casein-Units/ml of solution. Patients were required to rinse their mouths with the composition for 5 minutes twice a day for a period of no more than 7 days. The desirable dosage range was determined to be about 0.1 to 100 mg or 1 to 35 mg per treatment. In a related study, the composition was painted over the teeth and gingival of a dog twice a day until all the plaque was completely decomposed.

U.S. Patent Publication No. 2006/0134017 discloses an oral treatment composition for the retardation of bacterial plaque accumulation (See paragraph 4) containing an effective amount of an anti-adhesion protease enzyme, such as Krillase®, in an amount less than 100 parts by weight of the composition. Specifically, the Krillase® is present in an amount of at least 0.01-10 parts per 100 parts of the composition (See paragraphs 8-11). The composition may be formulated as a paste (See paragraphs 11 and 18). No examples including Krillase® are provided.

U.S. Patent Publication No. 2006/0140881 discloses an oral composition for reducing oral inflammation (See paragraph 10) and treating and/or inhibiting various oral conditions such as gingivitis and periodontitis. The composition may be formulated in a variety of different configurations among which are mentioned gums (See paragraphs 12 and 99). The compositions comprise an optional oral care active agent (See paragraph 13). In one embodiment, the oral care active agent may be a biofilm disruption agent such as protease enzymes including Krillase® (See paragraph 64-65). The oral care agent may be added in a safe and effective amount f from about 0.001% to about 5% by weight of the composition (See paragraph 76). No examples of gums or krill enzyme compositions are provided.

These references, however, do not disclose a suitably effective method for treating gingivitis using a krill enzyme composition. Thus, there remains a need for the development of more effective methods for the treatment of gingivitis and/or removal of dental plaque.

### SUMMARY OF THE INVENTION

One aspect of the present invention provides a method for treating disorders of the oral cavity using a composition comprising at least one krill enzyme, wherein the composition has a krill enzyme concentration of about 0.005 U/g to about 0.5 U/g, based on the total weight of the chewing gum composition.

Another aspect of the present invention provides a method for treating disorders of the oral cavity using a composition comprising at least one krill enzyme, wherein the composition has a krill enzyme concentration of about 0.005 U/g to about 0.5 U/g, based on the total weight of the chewing gum composition, and wherein the composition is formulated as a chewing a gum.

A third aspect of the invention provides a method for reducing plaque or accumulation of plaque in the oral cavity using a composition comprising at least one krill enzyme, wherein the composition has a krill enzyme concentration of about 0.005 U/g to about 0.5 U/g, based on the total weight of the chewing gum composition.

A fourth aspect of the present invention provides a method of reducing plaque or accumulation of plaque in the oral cavity using a composition comprising at least one krill enzyme, wherein the composition has a krill enzyme concentration of about 0.005 U/g to about 0.5 U/g, based on the total weight of the chewing gum composition, and is formulated as a chewing gum.

In another aspect, the present invention relates to a chewing gum composition which comprises 0.005 U/g to 0.5 U/g of krill enzyme, based on the total weight of the chewing gum composition.

These and other objects, advantages and aspects of the invention will become apparent in light of the following detailed description of certain embodiments of the invention.

### BRIEF DESCRIPTION OF THE DRAWINGS

FIG. 1 is a graph of the number of gingival bleeding sites for patients treated with a placebo gum composition, a 0.03 U/g (0.06 U) Krillase® gum composition and a 3 U/g (6 U) Krillase® gum composition.

FIG. 2a is a graph of the amount of plaque formation for patient 1 after being treated with a placebo gum composition, a 0.03 U/g (0.06 U) Krillase® gum composition and a 3 U/g (6 U) Krillase® gum composition.

FIG. 2b is a graph of the amount of plaque formation for patient 2 after being treated with a placebo gum composition, a 0.03 U/g (0.06 U) Krillase® gum composition and a 3 U/g (6 U) Krillase® gum composition.

FIG. 2c is a graph of the amount of plaque formation for patient 3 after being treated with a placebo gum composition, a 0.03 U/g (0.06 U) Krillase® gum composition and a 3 (6 U) U/g Krillase® gum composition.

FIG. 2d is a graph of the amount of plaque formation for patient 4 after being treated with a placebo gum composition, a 0.03 U/g (0.06 U) Krillase® gum composition and a 3 U/g (6 U) Krillase® gum composition.

FIG. 2e is a graph of the amount of plaque formation for patient 5 after being treated with a placebo gum composition, a 0.03 U/g (0.06 U) Krillase® gum composition and a 3 U/g (6 U) Krillase® gum composition.

FIG. 2f is a graph of the amount of plaque formation for patient 6 after being treated with a placebo gum composition, a 0.03 U/g (0.06 U) Krillase® gum composition and a 3 U/g (6 U) Krillase® gum composition.

FIG. 3 is a graph of enzyme activity as a function of time for a 0.03 U/g (0.06 U) Krillase® gum composition.

### DETAILED DESCRIPTION OF THE PREFERRED EMBODIMENTS

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not limited in its application to the details of any particular embodiment shown. The terminology used herein is for the purpose of description and not of limitation. Further, although certain methods are described with reference to certain steps that are presented herein in certain order, in many instances, these steps may be performed in any order as may be appreciated by one skilled in the art, and the methods are not limited to the particular arrangement of steps disclosed herein.

The invention is directed to chewing gum compositions and methods for treating, controlling and/or preventing disorders or diseases of the oral cavity such as aphtes, lesions, gum disease and dental diseases. The invention is specifically directed to a method for treating diseases or disorders of the oral cavity induced by accumulation of plaque by administering a composition comprising at least one krill enzyme in a concentration of from about 0.005 U/g to about 0.5 U/g of the chewing gum composition. Specifically, the composition of the present invention may be used to prevent, treat or control the development of gingivitis, gum disease, and/or symptoms thereof. Without wishing to be bound by theory, the composition appears to effectively prevent, treat and/or control the development of diseases or disorders of the oral cavity by reducing plaque or reducing or eliminating the accumulation of plaque and/or bio film.

The chewing gum composition of the present invention comprises at least one krill enzyme and a chewing gum base. Preferably, the composition is stable, biocompatible and capable of controlling and substantially sustaining enzymatic activity over a period of at least 1 hour upon initiation of chewing. Furthermore, the composition is preferably a non-irritant, allergen free, safe and effective oral treatment composition that conforms to EU/FDA pharmaceutical regulations.

The at least one krill enzyme of the present invention may be any suitable krill derived enzyme. Suitable krill enzymes are described in, for example, U.S. Pat. Nos. 6,524,814 and 5,958,406, the disclosures of which are hereby incorporated by reference for a description of suitable krill enzymes. Krill enzymes may also be derived from Antarctic krill (Euphausia superba) using a conventional separation and desorption method, or Krillase® may be used or any organism of the Euphausia genus as a krill enzyme source. In a preferred embodiment, the krill enzyme is a proteolytic enzyme. In an exemplary embodiment, the enzyme is Krillase®.

The effectiveness of the composition for treating, controlling and/or preventing diseases of the oral cavity induced by plaque is substantially dependent upon the concentration of krill enzyme in the composition. In general, a suitable krill enzyme concentration may range from about 0.005 U/g to about 0.5 U/g of the chewing gum composition, more preferably, about 0.005 U/g to about 0.25 U/g of the chewing gum composition, and most preferably about 0.005 U/g to about 0.05 U/g of the chewing gum composition, where one unit (U) of activity causes the liberation of 1 μmole of tyrosine from a tyrosine standard, per minute at 35° C.

Enzymatic activity and the effectiveness of the composition are greatest for compositions having lower krill enzyme concentrations. Without wishing to be bound by theory, the effectiveness of low krill enzyme concentrations may be explained by substrate-enzyme dependency. The effectiveness of the composition is therefore dependent upon a complex calculus that takes into consideration the amount of available enzyme, the amount of available substrates, the controlled release of the enzyme and enzymatic activity levels. It has been determined that compositions comprising low krill enzyme concentrations have greater enzymatic activity and are more effective in inhibiting and reducing the formation of dental plaque, inflammation of the gums and gingival bleeding as shown in the examples appended hereto. Furthermore, without wishing to be bound by theory, the present invention prevents and or reduces plaque formation, and consequently may be used to treat various peridontonal diseases, by disrupting dental bio film and limiting bacterial attachment.

The chewing gum compositions of the present invention may be in the form of a conventional chewing gum or any other product suitable for chewing. Suitable physical forms include sticks, dragees, chiclets, and batons. The chewing gum may also be a digestible or dissolvable gum suitable for chewing. In a preferred embodiment, the gum composition is anhydrous and formation does not require heating the krill enzyme composition to the point of denaturing the enzyme composition. A chewing gum is typically retained in the oral cavity for a time sufficient to allow ingredients released to contact substantially all of the dental surfaces and/or oral tissues for purposes of oral activity.

The chewing gum composition of the present invention may contain any conventional chewing gum base. Suitable chewing gum bases may contain an insoluble component and a soluble component. In a preferred embodiment, the gum comprises an anhydrous gum base.

The insoluble component of the gum base may comprises elastomers, resins, fats and oils, softeners and inorganic fillers. The gum base may or may not include wax. The insoluble component may constitute approximately 5% to about 95% by weight of the chewing gum, more commonly the gum base comprises 10% to about 50% of the gum, and in some preferred embodiments approximately 25% to about 35%, by weight, of the chewing gum.

In one embodiment, the insoluble component of the chewing gum base of the present invention contains about 20% to about 60% by weight synthetic elastomer, about 0% to about 30% by weight natural elastomer, about 5% to about 55% by weight elastomer plasticizer, about 4% to about 35% by weight filler, about 5% to about 35% by weight softener, and optional minor amounts (about 1% or less by weight) of miscellaneous ingredients such as colorants, antioxidants, etc.

Synthetic elastomers may include, but are not limited to, polyisobutylene with GPC weight average molecular weight of about 10,000 to about 95,000, isobutylene-isoprene copolymer (butyl elastomer), styrene-butadiene, copolymers having styrene-butadiene ratios of about 1:3 to about 3:1, polyvinyl acetate having GPC weight average molecular weight of about 2,000 to about 90,000, polyisoprene, polyethylene, vinyl acetate-vinyl laurate copolymer having vinyl laurate content of about 5% to about 50% by weight of the copolymer, and combinations thereof.

Preferred ranges for polyisobutylene are 50,000 to 80,000 GPC weight average molecular weight and for styrene-butadiene are 1:1 to 1:3 bound styrene-butadiene, for polyvinyl acetate are 10,000 to 65,000 GBC weight average molecular weight with the higher molecular weight polyvinyl acetates typically used in bubble gum base, and for vinyl acetate-vinyl laurate, vinyl laurate content of 10 45%.

Natural elastomers may include natural rubber such as smoked or liquid latex and guayule as well as natural gums such as jelutong, lechi caspi, perillo, sorva, massaranduba balata, massaranduba chocolate, nispero, rosindinha, chicle, gutta hang kang, and combinations thereof. The preferred synthetic elastomer and natural elastomer concentrations vary depending on whether the chewing gum in which the base is used is adhesive or conventional, bubble gum or regular gum, as discussed below. Preferred natural elastomers include jelutong, chicle, sorva and massaranduba balata.

Elastomer plasticizers may include, but are not limited to, natural rosin esters such as glycerol esters or partially hydrogenated rosin, glycerol esters of polymerized rosin, glycerol esters of partially dimerized rosin, glycerol esters of rosin, pentaerythritol esters of partially hydrogenated rosin, methyl and partially hydrogenated methyl esters of rosin, pentaerythritol esters of rosin; synthetics such as terpene resins derived from alpha-pinene, beta-pinene, and/or d-limonene; and any suitable combinations of the foregoing. The preferred elastomer plasticizers will also vary depending on the specific application, and on the type of elastomer which is used.

Fillers/texturizers may include magnesium and calcium carbonate, ground limestone, silicate types such as magnesium and aluminum silicate, clay, alumina, talc, titanium oxide, mono-, di- and tri-calcium phosphate, cellulose polymers, such as wood, and combinations thereof.

Softeners/emulsifiers may include tallow, hydrogenated tallow, hydrogenated and partially hydrogenated vegetable oils, cocoa butter, glycerol monostearate, glycerol triacetate, lecithin, mono-, di- and triglycerides, acetylated monoglycerides, fatty acids (e.g. stearic, palmitic, oleic and linoleic acids), and combinations thereof.

Colorants and whiteners may include FD&C-type dyes and lakes, fruit and vegetable extracts, titanium dioxide, and combinations thereof.

The base may or may not include wax. An example of a wax-free gum base is disclosed in U.S. Pat. No. 5,286,500, the disclosure of which is incorporated herein by reference.

In addition to a water insoluble gum base portion, a typical chewing gum composition also includes a water soluble bulk component and one or more flavoring agents. The water soluble portion can include bulk sweeteners, high intensity sweeteners, flavoring agents, softeners, emulsifiers, colors, acidulants, fillers, antioxidants, and other components that provide desired attributes.

Softeners are added to the chewing gum in order to optimize the chewability and mouth feel of the gum. The softeners, which are also known as plasticizers and plasticizing agents generally constitute between approximately 0.5% to about 15% by weight of the chewing gum. The softeners may include glycerin, lecithin, and combinations thereof. Aqueous sweetener solutions such as those containing sorbitol, hydrogenated starch hydrolysates, corn syrup and combinations thereof, may also be used as softeners and binding agents in chewing gum.

Bulk sweeteners include both sugar and sugarless components. Bulk sweeteners typically constitute about 5% to about 95% by weight of the chewing gum, more typically, about 20% to about 80% by weight, and more commonly, about 30% to about 60% by weight of the gum. Sugar sweeteners generally include saccharide-containing components commonly known in the chewing gum art, including but not limited to, sucrose, dextrose, maltose, dextrin, dried invert sugar, fructose, levulose, glactose, corn syrup solids, and the like, alone or in combination. Sugarless sweeteners include, but are not limited to, sugar alcohols such as sorbitol, mannitol, xylitol, hydrogenated starch hydrolysates, maltitol, and the like, alone or in combination

High intensity artificial sweeteners can also be used, alone or in combination, with the above. Preferred sweeteners include, but are not limited to, sucralose, aspartame, salts of acesulfame, altitame, saccharin and its salts, cyclamic acid and its salts, glycerrhizinate, dihydrochalcones, thaumatin, monellin, and the like, alone or in combination. In order to provide longer lasting sweetness and flavor perception, it may be desirable to encapsulate or otherwise control the release of at least a portion of the artificial sweetener. Such techniques as wet granulation, wax granulation, spray drying, spray chilling, fluid bed coating, coacervation, and fiber extension may be used to achieve the desired release characteristics.

Combinations of sugar and/or sugarless sweeteners may be used in chewing gum. Additionally, the softener may also provide additional sweetness such as with aqueous sugar or alditol solutions.

If a low calorie gum is desired, a low caloric bulking agent can be used. Examples of low caloric bulking agents include: polydextrose; Raftilose, Raftilin; Fructooligosaccharides (NutraFlora); Palatinose oligosaccharide; Guar Gum Hydrolysate (Sun Fiber); or indigestible dextrin (Fibersol). However, other low calorie bulking agents can be used.

A variety of flavoring agents can also be used, if desired. The flavor can be used in amounts of about 0.1 to about 15 weight percent of the gum, and preferably, about 0.2% to about 5% by weight. Flavoring agents may include essential oils, synthetic flavors or mixtures thereof including, but not limited to, oils derived from plants and fruits such as citrus oils, fruit essences, peppermint oil, spearmint oil, other mint oils, clove oil, oil of wintergreen, anise and the like. Artificial flavoring agents and components may also be used. Natural and artificial flavoring agents may be combined in any sensorially acceptable fashion. In order to maximize stability of the krill enzymes, which may be water soluble in the chewing gum base, it may be desirable to formulate a gum composition that contains up to about 5% by weight of water to enable sustained enzymatic activity. More preferably, the gum composition may contain up to about 3% by weight of water, and, most preferably, up to about 2% by weight of water. The maximum water content of a particular gum composition will depend on the desired enzymatic activity level and/or desired duration of enzymatic activity. It may also be desirable to include a base/emulsifer system and/or surfactant which leads to the desired concentration of the krill enzyme in the oral cavity so as to create a more hydrophilic balance. Furthermore, it may be desirable to include a component that enables the controlled release of said krill enzyme so as to control, sustain and optimize enzymatic activity.

Optionally, the composition may further comprise at least one of the following additives or a mixture thereof in order to enhance the performance of the krill enzyme or provide additional treatment benefits. In an preferred embodiment, zinc and/or urea may be desirable additives for potentiating enzyme activity. At least one anticalculus agent may also be optionally added, such as the one or more of the anticalculus agents recited in U.S. Pat. No. 5,292,526 to Gaffar et al., which is herein incorporated by reference. Preferably, the anticalculus agent is an anticalculus polyphosphate, at least one wholly or partially neutralized alkali metal, ammonium tripolyphosphate or hexametaphosphate salt present in the composition at an effective anticalculus amount, at least one water soluble, linear, molecularly dehydrated polyphosphate salt effective in an anticalculus amount or a mixture of potassium and sodium salts, at least one of which is present in an effective anticalculus amount as a polyphosphate anticalculus agent.

The anticalculus agent can also contain an effective anticalculus amount of linear molecularly dehydrated polyphosphate salt anticalculus agent present in a mixture of sodium and potassium salts. The ratio of potassium to sodium in the composition can be in the range of about 3:1, for example. The polyphosphate can be present in the oral composition in various amounts. An example of an oral composition comprises an oral care active agent, where the weight ratio of polyphosphate ion to an oral care agent ranges from in excess of about 0.7:1 to less than about 4:1, or wherein the weight ratio of the efficacy-enhancing agent to the polyphosphate ion ranges from about 1:6 to about 2.7:1, or wherein the weight ratio of the efficacy-enhancing agent to the polyphosphate ranges from about 1:6 to about 2.7:1. Other useful anticalculus agents include polycarboxylate polymers and polyvinyl methyl ether/maleic anhydride (PVME/MA) copolymers, such as GANTREZ®.

In order to optimize the anticalculus effectiveness of the oral composition, inhibitors against enzymatic hydrolysis of the polyphosphate may be a further additive to the composition. Such agents include a fluoride ion source sufficient to supply 25 ppm to 5,000 ppm or 25 ppm to 2,000 ppm of fluoride ions at an amount of about 0.01% to about 5% by weight, and 0% to 3% of a synthetic anionic polymeric polycarboxylate having a molecular weight of about 1,000 to about 1,000,000, preferably about 30,000 to about 500,000.

The compositions may further include an abrasive additive. Any orally acceptable abrasive can be used, but type, fineness (particle size) and amount of abrasive should be selected so that tooth enamel is not excessively abraded in normal use of the composition. Suitable abrasives include silica, for example in the form of silica gel, hydrated silica or precipitated silica, alumina, insoluble phosphates, calcium carbonate, resinous abrasives such as urea-formaldehyde condensation products, and mixtures thereof. Among insoluble phosphates useful as abrasives are water-insoluble orthophosphates, polymetaphosphates and pyrophosphates. Illustrative examples of these include dicalcium orthophosphate dihydrate, calcium pyrophosphate, .beta.-calcium pyrophosphate, tricalcium phosphate, calcium polymetaphosphate, and insoluble sodium polymetaphosphate. One or more abrasives are optionally present in an abrasive-effective total amount, typically about 5% to about 70%, for example about 10% to about 50% or about 15% to about 30% by weight of the composition. Average particle size of an abrasive, if present, is generally about 0.1 μm to about 30 μm, for example about 1 μm to about 20 μm, or about 5 μm to about 15 μm.

The chewing gum composition of the present invention is capable of treating, controlling and/or preventing diseases or disorders of the oral cavity, including aphtes, lesions, gum disease, dental diseases and diseases induced by plaque or biofilm. The composition of the present invention is particularly effective for treating, controlling or preventing gingivitis, gum disease, and/or symptoms thereof such as gingival bleeding. In one aspect, the krill enzyme may function as an anti-adhesion agent that hydrolyzes bio film components and/or disables oral bacteria from attaching to a surface of the oral cavity. By disrupting the dental bio film, i.e. pellicle, and inhibiting bacterial adherence to surfaces of the oral cavity, the composition deters the formation of plaque and development of various periodontal diseases. Specifically, it is believed that the present composition disintegrates bacterial surface adhesive proteins and hampers colonization of dental surfaces. Therefore, the maturation process of pathogenic plaque formation is disturbed, consequently decreasing the amount of plaque formation. However, a more complex interaction between the krill enzyme composition and substrates in the oral cavity cannot be excluded. Due to the rapid and highly efficient breakdown of complex biological substrates, such as dental plaque, accompanied by a safety profile which permits administration of various amounts and frequencies of administration, the composition of the present invention is highly effective for treatment and care of the oral cavity.

The method of the present invention involves administering to a patient a chewing gum composition of the present invention inorder to treat, control and/or prevent disorders or diseases of the oral cavity, particularly diseases or disorders induced by the accumulation of plaque. The method of the present invention may specifically be used to treat, control or prevent the development of gingivitis, gum disease, and/or symptoms thereof, such as gingival bleeding.

The gum composition is preferably chewed such that the gum is circulated around the oral cavity and substantially contacts the occlusal, buccal and lingual portions of the teeth of and upper and lower dental arches. More preferably, the gum contacts the gingival line during chewing.

For the most effective results, the composition of the present invention should be administered at least 2 times a day, more preferably, about 2 to about 8 times a day and most preferably, about 4 to about 6 times a day. The chewing gum composition is preferably chewed for a period of about 5 to about 20 minutes, more preferably, when chewed for a period of about 10 to 20 minutes and most preferably, when chewed for about 10-15 minutes per administration. Ideally, treatment should last for a period of 1-30 days, 1-14 days and most preferably 1-7 days in order to achieve significant results. In a preferred embodiment, a clinical effect on gingivitis may be expected after 7 days of chewing 4 times daily for at least 10 minutes.

After administration of the gum composition of the present invention, the incidence of contracting disorders or diseases of the oral cavity, including aphtes, lesions, gum disease, dental diseases and diseases or disorders induced by accumulation of plaque, such as gingivitis, gum disease, and/or symptoms thereof, may be reduced. Administration of the composition of the present invention may also be used to retard the development of these disorders or diseases. Furthermore, such administration may reduce the development of dental plaque by at least about 20%, and typically from 30-65%, more preferably by about 40%-60% and most preferably about 50%-60% and gingival bleeding by about 40%-65%, more preferably by about 40%-60% and most preferably about 40%-50%.

### EXAMPLES

### Example 1

A Krillase® containing chewing gum composition was found to be effective for inhibiting the formation of dental plaque and reducing gingival inflammation and bleeding. This example further established that frequent administration of low concentrations of Krillase® gum is most effective for reducing plaque build-up and inflammation of the gums.

In conjunction with standard oral hygiene measures, seven healthy male volunteers aged 21-45 were each administered one of a 3.0 U/g Krillase® gum composition, 0.03 U/g Krillase® gum composition and a placebo of peppermint gum during three 10 day cycles of the study. The Krillase® gum composition was formulated from a standard purified extract of proteolytic enzymes, including endo-peptidases and exo-peptidases isolated from Antarctic krill (Euphausia superba).

The test subjects were randomly provided one gum composition during each cycle and were required to chew the composition for at least 10 minutes after meals, 4 times a day, for 10 days, in conjunction with normal oral hygiene measures. Each test subject was randomly given a different gum composition prior to beginning each 10 day cycle of the study. Each participant participated in the study for three consecutive trial periods for each gum in a double-blind cross-over study. None of the subjects reported any adverse reactions or events.

The number of gingival bleeding sites was measured by gently probing gingival pockets from the first molar and forward in each jaw, initially and after each test cycle. The number of bleeding sites was recorded as bleeding on probing (BOP) in accordance with the Löe index 2 (See Löe H., “The gingival index, the plaque index and the retention index systems,” J. Periodontol, 38, 610-616, 1967). No participant was evaluated to have an index 3, which represents spontaneous bleeding, during the trial. In parallel, the plaque index was evaluated using the Löe index. The amount of plaque accumulated on the teeth of each test subject was initially evaluated using a plaque index having two index values and similarly after each test cycle. For each tooth the extent and amount of plaque was evaluated either as not visible plaque, index 1, or visible plaque, index 2, according to Löe. No participant reached index value 3, which represents voluminous plaque. Prior to evaluating the amount of plaque accumulation, one test subject left the study.

The tests were performed using the paired Wilcoxon signed rank test. If the data contained ties no exact p-values could be calculated, instead the p-values were obtained using normal approximation with continuity correction. All statistical comparisons are based on a 2-sided test using an alpha level of significance of 0.050. The paired tests have been performed partly comparing the two dosages of the Krillase® chewing gum (0.03 U/g and 3.0 U/g) with placebo and partly comparing the two dosages with each other. The percentage for placebo and the two Krillase® dosages is the ratio of the mean of the three treatments compared to the initial status mean.

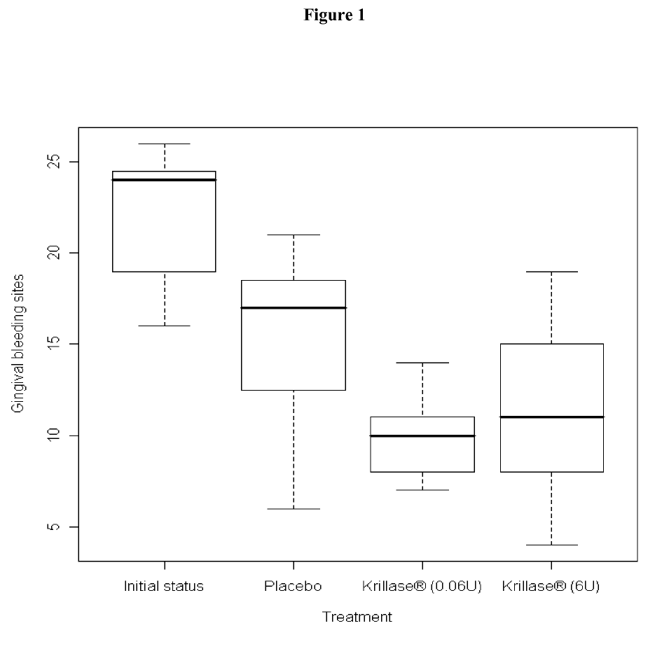

As shown in FIG. 1 and Tables 1-2, administration of the Krillase® gum compositions, in addition to employing standard oral hygiene techniques, significantly reduced the number of gingival bleeding sites in comparison to the administration of the placebo gum. The 0.03 U/g Krillase® gum composition was the most effective and reduced the greatest number of gingival bleeding sites.

TABLE 1
Total Gingival Bleeding Sites Counted After Each Treatment Regimen

| | Patient (no) | | | | | | |
|---|---|---|---|---|---|---|---|
| Treatment | 1 | 2 | 3 | 4 | 5 | 6 | 7 |
| Initial status | 25 | 24 | 16 | 24 | 22 | 16 | 26 |
| Placebo | 21 | 11 | 17 | 6 | 18 | 14 | 19 |
| Krillase ® | 8 | 10 | 10 | 7 | 8 | 14 | 12 |
| (0.03 U/g) | | | | | | | |
| Krillase ® | 12 | 4 | 8 | 8 | 18 | 11 | 19 |
| (3 U/g) | | | | | | | |

TABLE 2
Treatment Statistics Based on the Data in Table 1 for All Patients

| | | | | | p-value |
|---|---|---|---|---|---|
| | | Standard | % (of initial | p-value | (≠Krillase ® |
| Treatment | Mean | Deviation | status) | (≠placebo) | (0.03 U/g)) |
| Initial status | 21.86 | 4.18 | 100.0% | | |
| Placebo | 15.14 | 5.21 | 69.3% | — | |
| Krillase ® | 9.86 | 2.48 | 45.1% | 0.073 | — |
| (0.03 U/g) | | | | | |
| Krillase ® | 11.43 | 5.47 | 52.3% | 0.104 | 0.578 |
| (3 U/g) | | | | | |

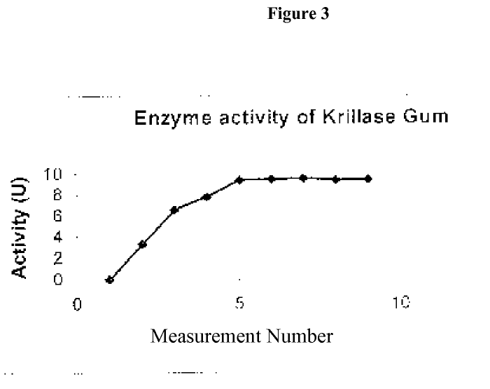

As shown in FIGS. 3(a)-3(f) and Tables 3-5, the 0.03 U/g Krillase® chewing gum composition reduced plaque formation significantly reduced plaque index 1 in comparison to that of the placebo gum composition.

TABLE 3
Plaque Accumulation After Each Treatment Regimen

| | |
|---|---|
| Plaque | Patient (no) |

| Treatment | Index | 1 | 2 | 3 | 4 | 5 | 6 |
|---|---|---|---|---|---|---|---|
| Initial status | 1 | 21 | 22 | 10 | 28 | 14 | 16 |
| | 2 | 5 | 2 | 22 | 1 | 0 | 7 |
| Placebo | 1 | 11 | 14 | 20 | 11 | 10 | 15 |
| | 2 | 0 | 0 | 4 | 0 | 0 | 2 |
| Krillase ® | 1 | 7 | 9 | 14 | 5 | 11 | 14 |
| 0.03 U/g | 2 | 1 | 0 | 5 | 0 | 0 | 0 |
| Krillase ® | 1 | 6 | 12 | 13 | 17 | 6 | 15 |
| 3 U/g | 2 | 0 | 0 | 5 | 1 | 0 | 0 |

TABLE 4
Treatment Statistics Based on the Data in Table 3 for All Patients

| | | | | | | p-value |
|---|---|---|---|---|---|---|
| | | | Std. | % (of initial | p-value | (≠Krillase ® |
| Treatment | | Mean | Dev. | status) | (≠placebo) | (0.03 U/g)) |
| Initial status | 1 | 18.50 | 6.44 | 100% | | |
| | 2 | 6.17 | 8.18 | 100% | | |
| Placebo | 1 | 13.50 | 3.73 | 73.0% | — | |
| | 2 | 1.00 | 1.67 | 16.2% | — | |
| Krillase ® | 1 | 10.00 | 3.69 | 54.1% | 0.073 | — |
| 0.03 U/g | 2 | 1.00 | 2.00 | 16.2% | 1.00 | — |
| Krillase ® | 1 | 11.50 | 4.59 | 62.2% | 0.419 | 0.832 |
| 3 U/g | 2 | 1.00 | 2.00 | 16.2% | 1.000 | 1.000 |

TABLE 5
Treatment Statistics for All Patients (Summation of Index 1 and 2)

| | | | | | p-value |
|---|---|---|---|---|---|
| | | | % (of initial | p-value | (≠Krillase ® |
| Treatment | Mean | SD | status) | (≠placebo) | (0.03 U/g)) |
| Initial status | 24.67 | 6.19 | 100% | | |
| Placebo | 14.50 | 5.32 | 58.8% | — | |
| Krillase ® | 11.00 | 4.94 | 44.5% | 0.058 | — |
| (0.03 U/g) | | | | | |
| Krillase ® | 12.50 | 5.50 | 50.7% | 0.400 | 0.916 |
| (3 U/g) | | | | | |

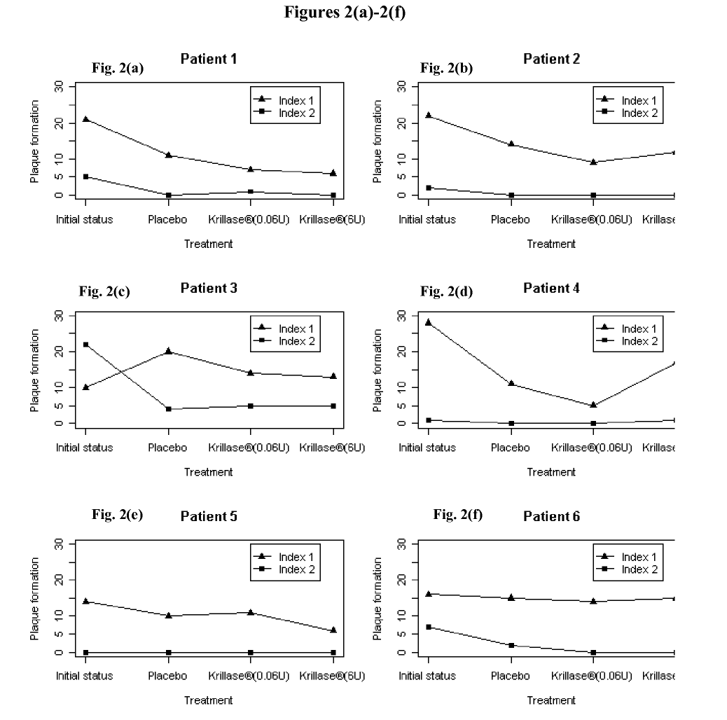

The clinical data clearly shows that a 0.03 U/g Krillase® gum composition is a therapeutically effective complement to normal oral hygiene for the purpose of reducing gingival bleeding sites, the amount of gingival bleeding and plaque accumulation. FIGS. 1 and 2(a)-(f) and Tables 1-5, which show a dramatic reduction in number of gingival bleeding sites and plaque build-up by chewing 0.03 U/g Krillase® gum, suggest that the Krillase® gum compositions disintegrate bacterial surface adhesive proteins and hampers colonisation of dental surfaces. Therefore the maturation process of pathogenic plaque formation is disturbed resulting in decrease of plaque formation.

The data also demonstrates the superiority of the Krillase® gum compositions in comparison to placebo gum compositions. Although the placebo did effectively diminish the number of gingival bleeding sites and plaque accumulation, this is attributed to the mechanical effect of chewing gum as well as to the known placebo effect phenomenon of unconsciously increasing oral hygiene awareness triggered by participation in a dental trial. Notably, the p-values indicate that there is a statistically significant difference between the level of reduction in gingival bleeding sites and plaque accumulation in comparison to the reduction resulting from administration of the placebo, thereby affirming the conclusion that the Krillase® gum compositions are more effective than the placebo.

Tables 2 and 4-5 also demonstrate that gum compositions having high Krillase® concentrations do not improve efficacy; the 0.03 U/g Krillase® gum composition was found to be more effective in reducing the number of gingival bleeding sites and plaque accumulation than that of the 3 U/g Krillase® gum composition.

### Example 2

In comparison to the a placebo gum having no krill enzyme component, a 0.03 U/g Krillase® gum composition was found to significantly decrease the amount of voluminous plaque and gingival bleeding over a period of about 5 days as well as over a long term period of about 14 days.

In a double blind crossover pilot study, seven patients were alternatively administered a 0.03 U/g Krillase® gum composition and a placebo gum composition. Neither the patients nor the dentists were informed as to which gum composition each patient was administered throughout the trial. At the beginning of the study, the patients' teeth were professionally cleaned prior to administration of either the placebo or Krillase® gum composition. Each patient was then instructed to chew 1 piece of a designated gum four times a day for 10 min after meals for a period of 5, 10 or 14 days. During this period, patients were prohibited from engaging in any other oral hygiene measures, i.e. no brushing, toothpaste, flossing, tooth picking, rinsing nor other prophylactic devices such as chewing gums, rinses, tablets, lozenges and/or products containing fluoride, carbamide, xylitol, chlorhexidine etc. The patients were also instructed to eat normally. After a first 5 day test period, the base line was restored, and the patient began a new 10 day test period. The same procedure was than repeated for the third 14 day test period.

Plaque index (PLI), gingival bleeding on probing (BOP) and photographs were performed after each the test period and compared with the baseline values (Löe, 1967). In PLI, 0=no plaque, 1=no visible, but can be scraped, 2=visible and 3=voluminous plaque. In BOP, 0=healthy ginigiva, 1=mildly inflamed, no bleeding, 2=bleeding on probing and 3=spontaneous bleeding. The PLI and BOP results after a first washout period was designated as healthy (0). At the end of the 5, 10 and 14 day test periods, the patient's teeth were dyed with the coloring agent Diaplaque® and clinically evaluated for plaque build-up and gingival bleeding. Generation of a baseline and standard trial procedures were performed for both the placebo and Krillase® gum composition.

In addition to subjective clinical evaluations, 7 independent observers, namely 3 dentists, 1 hygienist and 3 doctors, also assessed blinded photographs and graded the plaque extension in three randomly selected patients. Further evaluations were performed by advanced computerized image analyses to objectively quantify the color differences in the test photographs. The color differences were ascertained by including a reference mm/gray scale in every photograph as an internal standard. Based on the reference scale, the photographs were corrected both for the geometric and photometric distortions. Thus, the reference gray scale in the photos makes it possible to recreate the original color reflectance values in each pixel in spite of variations in color temperature of the illumination, photographic emulsion and other factors, which cannot be fully controlled. In this manner all photographs were “normalized” before analyses. The resulting data therefore was an accurate account of plaque extension in particular tooth.

The clinical data, summarized in Table 6 clearly show the superiority of Krillase® gum composition in comparison to the placebo gum. The Krillase® gum composition was found to be effective for both short as well as long term treatment periods for both PLI and BOP. Further evaluations by independent observers, as shown in Table 7, confirmed these findings. Also complementary computerized analyses further supported the clinical evaluations. As shown in Table 8, Placebo treated teeth's were found to present considerably higher amounts of plaque in comparison to Krillase®. The plaque thickness was evaluated in terms of the percent extension on each tooth.

TABLE 6
Clinical Evaluation of Plaque Index and Bleeding

| | Treatment | Krillase ® | | Placebo | | Chx chew g | | Denivit | | Colg 2-1 | |
|---|---|---|---|---|---|---|---|---|---|---|---|
| Case | (days) | PLI | BOP | PLI | BOB | PLI | BOP | PLI | BOP | PLI | BOP |
| A | 5 | 1 | 1 | 3 | 2 | | | | | | |
| A | 14 | 2 | 1 | 3 | 3 | | | | | | |
| B | 5 | 1 | 1 | 3 | 2 | | | | | | |
| B | 14 | 2 | 2 | 3 | 3 | | | | | | |
| C | 10 | 2 | 1 | 3 | 2 | | | | | | |
| D | 10 | 1 | 1 | 2 | 2 | | | | | | |
| E | 10 | 1 | 1 | 2 | 2 | | | | | | |
| F | 10 | | | | | 2 | 2 | | | | |
| G | 10 | | | | | 2 | 2 | | | | |
| H | 10 | | | | | | | 2 | 2 | | |
| I | 10 | | | | | | | | | 2 | 2 |
| J | 10 | | | | | | | 1 | 1 | | |
| K | 10 | | | | | | | | | 2 | 2 |
| L | 10 | | | | | 2 | 1 | | | | |
| M | 10 | | | | | 2 | 1 | | | | |

TABLE 7
Multi-Observer Plaque Assessments Based on Dental Photographs
Taken After Different Treatments Over a 5 Day Treatment Period

Plaque extension

| Patient no | Observers | Low | Medium | High |
|---|---|---|---|---|
| 1 | I | Krillase ® | | Placebo |
| 2 | | Krillase ® | | Placebo |
| 3 | | Krillase ® | | Placebo |
| 1 | II | Krillase ® | | Placebo |
| 2 | | Krillase ® | | Placebo |
| 3 | | Krillase ® | | Placebo |
| 1 | III | Krillase ® | | Placebo |
| 2 | | Krillase ® | | Placebo |
| 3 | | Krillase ® | | Placebo |
| 1 | IV | | Krillase ® | Placebo |
| 2 | | Krillase ® | | Placebo |
| 3 | | Krillase ® | | Placebo |
| 1 | V | | Krillase ® | Placebo |
| 2 | | Krillase ® | | Placebo |
| 3 | | Krillase ® | | Placebo |
| 1 | VI | Krillase ® | | Placebo |
| 2 | | Krillase ® | | Placebo |
| 3 | | | Krillase ® | Placebo |
| 1 | VII | | Krillase ® | Placebo |
| 2 | | Krillase ® | | Placebo |
| 3 | | Krillase ® | | Placebo |

Clinical evaluations and standardized computerized analyses showed that the Krillase® gum composition, in comparison to the placebo, was more efficient in reducing plaque formation than placebo over a short term period of 5 days and over a long term of 10 days or 14 days. Patients reported no adverse effects, and patients also reported that their mouth felt “fresher” after chewing the Krillase® gum composition.

TABLE 8
Data From Computerized Image Analyses of Dental
Photographs Taken After Different Treatments

| Treatment | Tooth | Thick plaque | |
|---|---|---|---|
| (days) | (no) | extension (%) | Treatment |
| 5 | 1 | 2.2 | Krillase ® |
| | 2 | 1.1 | |
| | 3 | 0.4 | |
| | 5 | 1.3 | ö |
| | 6 | 0.0 | |
| | 7 | 0.8 | |
| | 8 | 0.0 | |
| 5 | 1 | 44.4 | Placebo |
| | 2 | 12.9 | |
| | 3 | 10.4 | |
| | 4 | 38.0 | |
| | 5 | 23.8 | |
| | 6 | 13.2 | |
| | 7 | 14.3 | |
| | 8 | 26.0 | |
| 5 | 1 | 27.5 | Placebo |
| | 2 | 7.2 | |
| | 3 | 7.4 | |
| | 5 | 8.9 | |
| | 6 | 2.7 | |
| | 7 | 4.0 | |
| | 8 | 5.6 | |
| 14 | 1 | 5.7 | Krillase ® |
| | 2 | 1.7 | |
| | 3 | 1.7 | |
| | 4 | 26.7 | |
| | 5 | 0.0 | |
| | 6 | 0.0 | |
| | 7 | 0.0 | |
| | 8 | 18.2 | |
| 14 | 1 | 64.3 | Placebo |
| | 2 | 21.2 | |
| | 3 | 42.2 | |
| | 4 | 58.6 | |
| | 5 | 44.9 | |
| | 6 | 58.3 | |
| | 7 | 48.9 | |
| | 8 | 62.4 | |
| 14 | 1 | 15.4 | Krillase ® |
| | 2 | 1.7 | |
| | 3 | 3.9 | |
| | 5 | 0 | |
| | 6 | 0.0 | |
| | 7 | 0.0 | |
| | 8 | 5.0 | |
| 14 | 1 | 87.5 | Placebo |
| | 2 | 41.1 | |
| | 3 | 43.9 | |
| | 5 | 80.9 | |
| | 6 | 75.8 | |
| | 7 | 72.0 | |
| | 8 | 77.9 | |
| 14 | 1 | 21.0 | Placebo |
| | 2 | 6.0 | |
| | 3 | 7.8 | |
| | 4 | 37.3 | |
| | 5 | 7.9 | |
| | 6 | 5.6 | |
| | 7 | 11.3 | |
| | 8 | 19.5 | |

### Example 3

A study of the enzymatic activity for the 0.03 U/g Krillase® gum composition of Example 1 demonstrates a controlled and gradual release the krill enzyme upon simulated chewing. For FIG. 3, the first measurement was taken after 5 minutes of simulated chewing and each subsequent measurement was taken after an additional two minutes of simulated chewing for measurements 2-9.

The Krillase® gum composition was cut into small pieces which were placed in 10 ml of Hepes buffer, having a pH of about 7.2 containing 1 mM CaCl₂. Enzyme activity was measured using trypsin specific protease substrate CBZ-pNA at a pH of about 8.0 at about 25° C. The first measurement was performed about 5 min after the gum pieces were placed in the buffer solution without mashing or stirring. Thereafter, to duplicate the effects of chewing, the gum, pieces were mashing by pushing a glass rod on the gum pieces, as a substitute for chewing. The gum composition was mashed 8 consecutive times over a period of about 5 minutes prior to the first measurement and then measurements 2-9 were taken at about 2 minute intervals thereafter.

FIG. 3 shows the release profiles of the krill enzymes from the chewing gum composition. Essentially no enzyme activity could be detected when gum pieces were simply placed in a solution. In comparison, full enzymatic activity was measurable after mashing.

It is to be understood that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meaning of the terms in which the appended claims are expressed.

1. A method for treating an oral cavity comprising the step of: chewing a chewing gum composition comprising at least one krill enzyme for a period of at least ten minutes per day, and wherein said chewing gum composition has a krill enzyme concentration of about 0.005 U/g to about 0.5 U/g of the gum composition.

2. The method of claim 1, wherein said chewing gum composition has a krill enzyme concentration of about 0.005 U/g to about 0.25 U/g of the gum composition.

3. The method of claim 1, wherein said chewing gum composition has a krill enzyme concentration of about 0.005 U/g to about 0.05 U/g of the gum composition.

4. The method of claim 1, wherein said chewing gum composition is administered 2 to 8 times per day.

5. The method of claim 1, wherein said chewing gum composition is administered 2 to 6 times per day.

6. The method of claim 1, wherein said composition further comprises a component selected from the group consisting of: at least one component that controls enzymatic release, at least one anticalculus agent, at least one abrasive agent and mixtures thereof.

7. The method of claim 1, wherein said method reduces plaque accumulation in said oral cavity.

8. The method of claim 1, wherein said method treats gingivitis.

9. The method of claim 1, wherein said method reduces gingival bleeding.

10. The method of claim 1, wherein said chewing gum composition is chewed for a period of about 5 to about 20 minutes per administration.

11. The method of claim 1, wherein said chewing gum composition is chewed for a period of about 10 to about 20 minutes per administration.

12. The method of claim 1, wherein said chewing gum composition is chewed for a period of about 10 to about 15 minutes per administration.

13. The method of claim 1, wherein said chewing gum composition further comprises a component that controls enzymatic release and wherein said chewing gradually releases the enzyme over a chewing period.

14. A chewing gum composition for treating plaque build-up comprising:
a. at least one krill enzyme having a concentration of about 0.005 U/g to about 0.5 U/g of the gum composition; and
b. a chewing gum base,
wherein said chewing gum composition is effective for treating gum disease or gingivitis.

15. The composition of claim 14, wherein said krill enzyme concentration is about 0.005 U/g to about 0.25 U/g of the gum composition.

16. The composition of claim 14, wherein said krill enzyme concentration is about 0.005 U/g to about 0.05 U/g of the gum composition.

17. The composition of claim 14, wherein said chewing gum base comprises a component that enables a controlled release of said krill enzyme.

18. The composition of claim 14, further comprising an ingredient selected from: at least one anticalculus agent, at least one abrasive agent, at least one antioxidant and a mixture thereof.

19. A chewing gum composition for treating plaque build-up comprising:
a. at least one krill enzyme having a concentration of about 0.005 U/g to about 0.05 U/g;
b. a chewing gum base comprising an elastomer, a plasticizer and an emulsifier; and
c. a component that enables a controlled release of said krill enzyme,
wherein said chewing gum composition is effective for treating gum disease or gingivitis.

20. The method of claim 19, wherein said composition is capable of reducing dental plaque and gingival bleeding by about 50%-60%.
